(19) United States
(12) Patent Application Publication
(10) Pub. No.: US 2026/0182975 A1
(43) Pub. Date: Jul. 2, 2026

(54) ENDOVASCULAR SNARE APPARATUS

(71) Applicant: BARD PERIPHERAL VASCULAR, INC., Franklin Lakes, NJ (US)

(72) Inventors: Andrzej J. Chanduszko, Chandler, AZ (US); Alexander Lastovich, Gilbert, AZ (US)

(21) Appl. No.: 19/130,029

(22) PCT Filed: Nov. 18, 2022
(86) PCT No.: PCT/US2022/080108

§ 371 Date: May 14, 2025

(51) Int. Cl. A61B 17/00; A61B 17/22; A61B 17/221

(52) U.S. Cl. CPC A61B 17/00234; A61B 17/221; A61B 2017/00358; A61B 2017/00367; A61B 2017/00477; A61B 2017/00526; A61B 2017/22035; A61B 2017/2212

(57) ABSTRACT

An endovascular snare apparatus having an outer tube with a tube wall surrounding a tube bore or lumen wherein a specially configured connection joins an end of a snare wire or cable to the tube wall wherein all or part of the connected end is located between the tube outer surface and the tube inner surface. The snare wire or cable occupies the outer tube bore or lumen, but a loop portion extends distally of the outer tube in an extended position that forms a snare loop. The snare loop can be opened or closed by advancing and retracting the snare wire or cable. The snare can be used for capturing and retrieving implants or other objects in vasculature. Since only one strand is needed to control the opening of the snare loop, the profile of the snare can be significantly reduced.

### CROSS-REFERENCE TO RELATED APPLICATIONS

Not applicable

### STATEMENT REGARDING FEDERALLY DEVELOPMENT SPONSORED RESEARCH OR

Not applicable

### REFERENCE TO A “MICROFICHE APPENDIX”

Not applicable

### BACKGROUND OF THE INVENTION

### 1. Field of the Invention

The present invention relates to endovascular snares and more particularly to an endovascular snare apparatus having an outer tube with a tube wall surrounding a tube lumen wherein a specially configured connection joins an end of a snare loop wire (one or more strands or cables) to the outer tube wall wherein all or a majority of the connected end of the snare loop wire is located between the tube outer surface and the tube inner surface.

### 2. General Background of the Invention

Endovascular snares have traditionally been used to retrieve vena cava filters, reposition indwelling catheters, fibrin sheath stripping, assist in central venal venipuncture and retrieve or adjust other foreign objects. Some examples of commercial snares include the Merit ONE Snare® Endovascular Snare System and Bard® Snare Retrieval Kit nitinol snare.

These snares require a separate snare sheath where the snare is collapsed during tracking and delivery. Once at the intended location, the snare is advanced out of this snare sheath. The sheath is also needed to collapse the snare, but the sheath also significantly increases the snare profile.

The following U.S. Patents, which are incorporated herein by reference, are possibly relevant.

| PATENT/ | | ISSUE |
|---|---|---|
| PUBLICATION NO. | TITLE | DATE |
| 5,387,219 | MEDICAL RETRIEVAL SNARE WITH COIL | Feb. 7, 1995 |
| | WRAPPED LOOP | |
| 6,517,550 | FOREIGN BODY RETRIEVAL DEVICE | Feb. 22, 2003 |
| 6,554,842 | SMALL DIAMETER SNARE | Apr. 29, 2003 |
| 7,520,881 | LOOP TIE WIRE GUIDE | Apr. 21, 2009 |
| 7,655,013 | SNARE RETRIEVAL DEVICE | Feb. 2, 2010 |
| 8,852,204 | MEDICAL SNARE DEVICE | Oct. 7, 2014 |
| 9,198,664 | TISSUE LIGATION DEVICES AND CONTROLS | Dec. 1, 2015 |
| | THEREFORE | |
| 9,827,008 | SNARE WITH LOOP MADE OF HEAT | Nov. 28, 2017 |
| | SHRINKABLE SHAPE MEMORY MATERIAL | |
| | AND METHOD OF USE THEREOF | |
| 11,013,533 | IMPLANT DELIVERY AND RETRIEVAL | May 25, 2021 |
| | SYSTEMS AND METHODS | |
| 2005/0234474 | SMALL-DIAMETER SNARE | Oct. 20, 2005 |
| 2006/0100544 | STEERABLE LOOP TIE WIRE-GUIDE | May 11, 2006 |
| 2006/0100545 | LOOP TIE WIRE GUIDE | May 11, 2006 |
| 2014/0364866 | ENDOSCOPIC SNARE DEVICE | Dec. 11, 2014 |
| 2020/0197151 | MULTI-ACCESS INTRAPROCEDURAL | Jun. 25, 2020 |
| | EMBOLIC PROTECTION DEVICE | |

### BRIEF SUMMARY OF THE INVENTION

The snare apparatus of the present invention is a low profile (e.g., 0.035″ outer diameter) snare that can also be used as a guidewire. The apparatus includes an outer tube (e.g., hypotube) with a diameter (e.g., between 0.025″ and 0.045″ inches (0.64 mm and 1.1 mm)) roughly the same as commercial snares without the need for an outer sheath.

An inner cable is a snare cable that can be comprised of multiple strands (e.g., three (3) strands as seen in FIGS. 2-3, 7 and 9, and seven (7) strands in FIGS. 4-5). The cross-section of all strands can match the one strand of current commercial snares, therefore providing equivalent tensile strength. A three (3) or seven (7) strand cross-section may be used as examples. Optionally, other configurations may be used, such as four, twelve or nineteen strands.

The strands may be welded together at a non-movable end that occupies all or part of a section of the tube or hypotube wall in between tube wall inner and outer surfaces (see FIGS. 2 and 5). In one embodiment, this can be accomplished by forming (e.g., laser cutting) a suitable opening/window in the tube or hypotube wall (e.g., see FIGS. 1 and 6), positioning the wires of the distal or free end of the snare over a mandrel 35 in the tube bore or lumen and welding strands to each other and to the tube or the hypotube wall (e.g., see FIG. 9). The strands could also be connected to the outer tube by soldering or brazing (adhesive may work but not very likely). Optionally, microholes can be drilled in the tube wall (e.g. using micro-EDM (Microelectrical discharge machining)) and snare strands can be inserted and welded (see www.sciencedirect.com/topics/engineering/microhole for examples). A tube including the formed opening before welding/assembly is shown in FIG. 6. A loop section of the snare cable can be extended distally of the outer tube. A proximal section of the snare cable occupies the outer tube lumen extending proximally of the tube lumen. While the distal or loop section of the snare cable can be comprised of multiple strands for increased flexibility, the straight or proximal section of the snare cable can be solid (e.g., a single thick wire or a tube) for improved pushability (e.g., see FIG. 12). Optionally, the whole length can be a single wire or multiple strands/cable form (see FIG. 12). Because the distal or free end of the snare member is connected to the tube wall as seen in FIGS. 2, 4, 5 and 9, a majority or all of the tube lumen is available to hold the snare member distal section. The snare member distal section thus has a diameter between fifty and one hundred percent (50%-100%) of the tube lumen.

The snare loop apparatus of the present invention can be opened or closed by advancing and retracting a proximal end portion of the snare loop by a physician or technician. The snare can be used for capturing and retrieving implants or other objects in vasculature. Since only one strand is needed to control the opening of the snare loop, the profile of the snare can be significantly reduced. A free or distal end of the snare cable attaches to the outer tube wall (FIGS. 2, 4 and 9) so that the outer tube lumen only need to provide room for a single snare cable section (comprised of one or multiple strands) extending through the lumen (FIGS. 2 and 4).

Different strand number and strand arrangements can be used. While the loop section (distal section) of the snare can be made of multiple strands for increased flexibility, the straight proximal section can be solid (e.g., a single thick wire, a tube or tube/thick wire combination) for improved pushability. Both proximal and distal sections of the snare cable can be joined, for example, with a weld or crimp (if hypotube is used). The whole snare length can be a single wire or multiple strands (cables). Radiopaque markers can be incorporated into the design such as on the snare loop or tube distal end.

Multiple snare loops can be incorporated into the design and can be separately activated as needed; this may be helpful when an object is difficult to snare due to the two (2) dimensional nature of x-ray-based imaging. The present invention eliminates the snare sheath, which reduces profile (e.g., approximately 25%) and potentially cost. The present invention reduces the profile of the introducer sheath since the snare profile is reduced. The present invention can eliminate the need for a separate snare/guidewire product code reducing procedural costs and simplifying inventory.

The present invention provides an endovascular snare apparatus. In one set of embodiments, the apparatus may comprise: an outer tube having a proximal end, a distal end, an outer surface, an inner surface, a tube lumen having a central, longitudinal axis and an outer tube wall in between the inner and outer surfaces; a snare member that includes one or more snare wires or cables that occupy the tube lumen, said one or more snare wires or cables having a snare proximal end and a snare distal end; the snare member having an outer diameter; wherein a majority of the snare member outer diameter is positioned in between the tube inner and outer surfaces at a connection of the snare distal end to the outer tube; wherein at said connection, a portion of said snare member is extendable distally of said tube distal end to form a snare loop; and a proximal portion of said snare member extending through said tube lumen to said tube proximal end so that a user can manipulate the snare member proximal portion to extend or retract said loop.

In one or more embodiments, the endovascular snare apparatus further comprises a recess in the outer tube distal end wherein the snare distal end can occupy said recess at said connection.

In one or more embodiments, the snare member can include multiple strands.

In one or more embodiments, the snare member can be bonded to the outer tube at the recess. The bonding can be accomplished with a weld, adhesive or brazing or soldering methods, for example.

In one or more embodiments, there can be between three and nineteen strands that comprise said snare member. For example, there can be between three and seven strands that comprise the snare member.

In one or more embodiments, each strand extends to the tube outer surface at the connection.

In one or more embodiments, each strand extends to the tube inner surface at the connection.

In one or more embodiments, the endovascular snare apparatus snare member can have a proximal section that is a single strand.

In one or more embodiments, the endovascular snare apparatus snare member can have a proximal section that is a tube.

In another set of embodiments, the present invention provides an endovascular snare apparatus, comprising: an outer tube having a proximal end, a distal end, an outer surface, an inner surface, an outer tube lumen having a central, a lumen interval diameter and a longitudinal axis and an outer tube wall in between the inner and outer surfaces; a snare member that includes one or more snare wires or cables that occupy the tube lumen, said one or more snare wires or cables having a proximal end and a distal end; the snare member having an outer diameter, and proximal and distal ends; wherein a majority of the outer diameter is positioned in between the tube inner and outer surfaces at a connection of the snare member distal end to the outer tube; wherein at said connection, a portion of said snare member extends distally of said tube distal end to form a snare loop, said loop comprised of multiple strands that each form a separate single strand loop; a proximal portion of said snare member extending through said tube bore to said tube proximal end so that a user can manipulate the snare member proximal portion to extend or retract said loop; and said proximal portion having a diameter that is more than half the diameter of the said tube lumen.

In one or more embodiments, the endovascular snare apparatus has between three and nineteen strands that comprise the snare member.

In one or more embodiments, the snare apparatus further comprises a recess in the outer tube distal end and the snare member distal end can occupy the recess.

In one or more embodiments, the snare member of the apparatus is bonded to the outer tube at the recess. The bonding with a weld, adhesive or brazing or soldering methods.

In one or more embodiments, each strand extends to the tube outer surface at the connection.

In one or more embodiments, each strand extends to said tube inner surface at the connection.

In one or more embodiments, each strand is generally parallel to the tube bore longitudinal axis at the connection, i.e. each strand deviates at most 10° from the tube bore longitudinal axis at the connection.

In one or more embodiments, multiple strands are bonded to each other and to the tube wall at the connection.

In yet another set of embodiments, the present invention provides an endovascular snare apparatus, comprising: an outer tube having a proximal end, a distal end, an outer surface, an inner surface, an outer tube lumen having a central, longitudinal axis and an outer tube wall in between the inner and outer surfaces; a snare member that includes one or more snare wires or cables that occupy the tube lumen, said one or more snare wires or cables having a proximal end and a distal end; the snare member having an outer diameter, and proximal and distal ends; wherein a majority of the outer diameter is positioned in between the tube inner and outer surfaces at a connection of the snare member distal end to the outer tube; wherein at said connection, a portion of said snare member extends distally of said tube distal end to form a snare loop, said loop comprised of multiple strands, each strand forming a separate single strand loop; a proximal portion of said snare member extending through said tube bore to said tube proximal end so that a user can manipulate the snare member proximal portion to extend or retract said loop; and wherein each strand extends a majority of the distance between said tube inner and outer surfaces.

In one or more embodiments, the endovascular snare apparatus snare member proximal portion has a transverse cross section that occupies between 50-100% percent of the tube lumen.

In one or more embodiments, the recess of the outer tube has small holes or spaces and the snare member is bonded to the outer tube at said small holes or spaces.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a side view of a preferred embodiment of the apparatus of the present invention;

FIG. 2 is a sectional view taken along lines 2-2 of FIG. 1;

FIG. 3 is a sectional view taken along lines 3-3 of FIG. 1;

FIG. 4 is a schematic diagram that compares the profile of a prior art snare with the profile of the snare apparatus of the present invention;

FIG. 5 is a cutaway end view of preferred embodiment of the apparatus of the present invention;

FIG. 6 is a partial side view of a preferred embodiment of the apparatus of the present invention;

FIG. 7 is a partial side view of a preferred embodiment of the apparatus of the present invention;

FIG. 8 is a sectional view taken along lines 8-8 of FIG. 6;

FIG. 9 is a cutaway end view of a preferred embodiment of the apparatus of the present invention illustrating use of a mandrel when welding strands to the outer tube;

FIG. 10 is a side view of a preferred embodiment of the apparatus of the present invention showing a deployed position;

FIG. 11 is a side view of a preferred embodiment of the apparatus of the present invention showing a retracted position; and

FIG. 12 is a partial side view of a preferred embodiment of the apparatus of the present invention showing a proximal section in the form of a single wire or hypotube and a distal section of multiple strands.

### DETAILED DESCRIPTION OF THE INVENTION

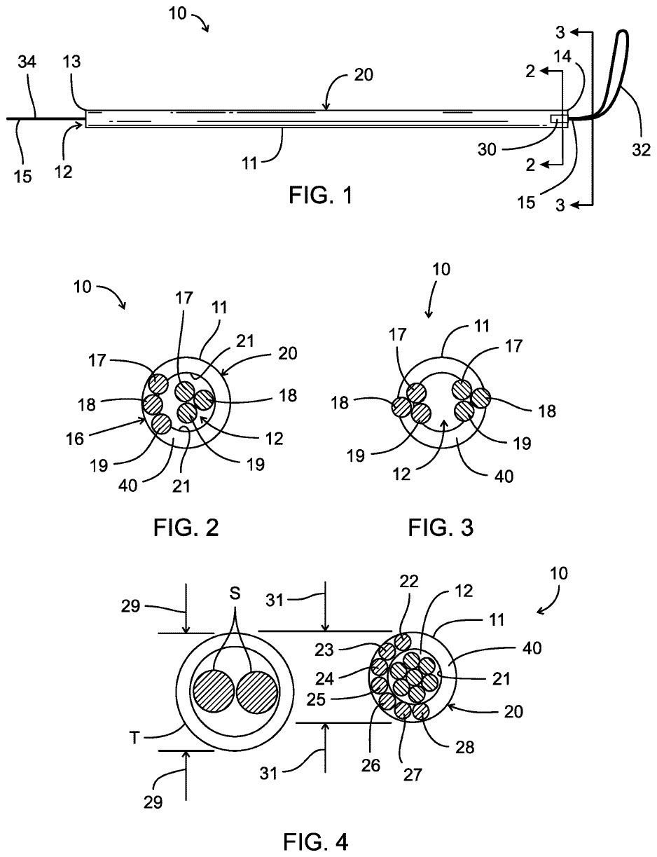

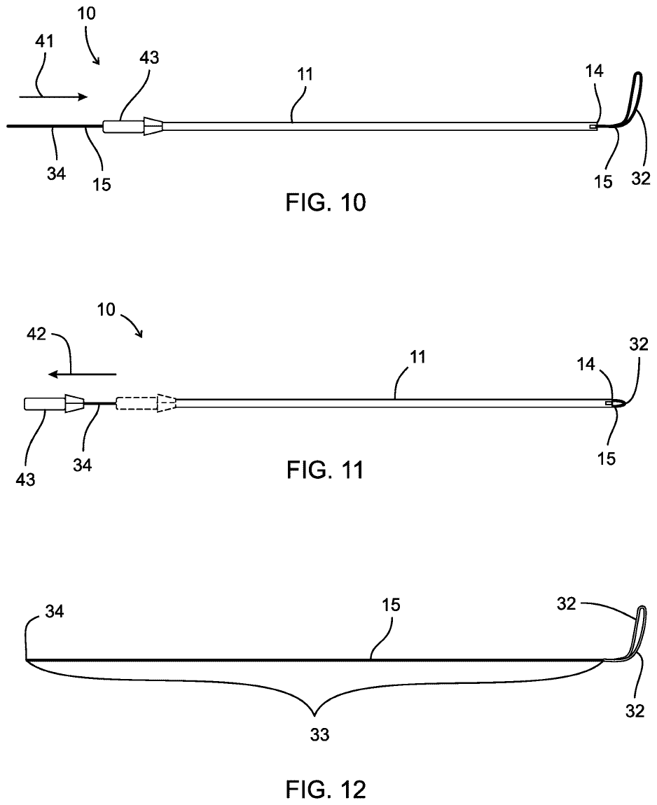

FIGS. 1-12 show a preferred embodiment of the apparatus of the present invention, designated generally by the numeral 10. Endovascular snare apparatus 10 includes an outer tube (e.g., hypotube) 11 having a tube wall 40 surrounding an elongated bore or lumen 12 (see, e.g., FIGS. 2-3). Tube 11 has proximal end 13 and distal end 14 (see FIG. 1). Bore 12 can extend from proximal end 13 to distal end 14. Snare member 15 occupies both proximally and distally of tube 11 bore/lumen 12 as seen in FIGS. 10-11.

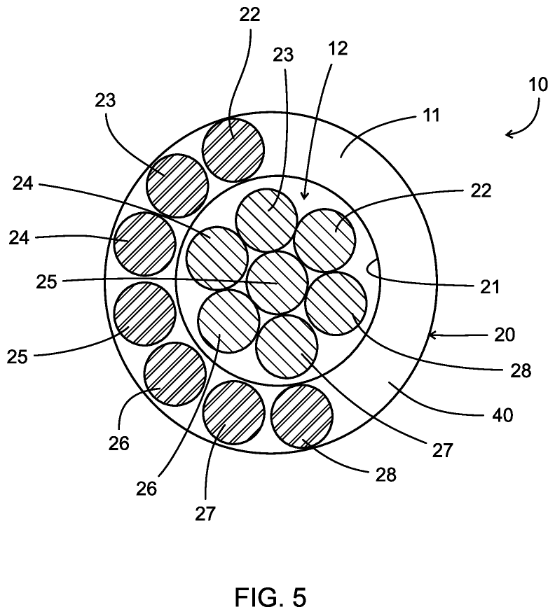

Snare member 15 can include multiple cables or wires such as the three (3) strands, cables or wires 17, 18, 19 in FIGS. 2, 3, 7 and 9 or seven strands 22-28 as seen in FIGS. 4 and 5. The snare member 15 includes a distal portion that can form a loop 32 (see, e.g., FIGS. 1, 10). The snare member 15 includes a proximal section 34 that enables a physician to enlarge and advance loop 32 (FIGS. 1, 10, 12) or retract the loop 32 (FIG. 11) by manipulating proximal section 34. The loop 32 is enlarged when a physician pushes section 34 distally (FIG. 10, arrow 41). FIG. 12 illustrates that a length 33 of snare member 15 extends from snare loop 32 in the extended position to proximal section 34. Length 33 of snare member 15 (the straight or proximal section of the snare cable) can be solid (e.g., a single thick wire or a tube) for improved pushability. In certain embodiments, a hypotube may be utilized for the straight or proximal section; in such embodiments where a hypotube is in place for the inner wire, the embodiment would appear similar to FIG. 2 but with a tube around the strands 17, 18, and 19 in the lumen.

The loop 32 is retracted when a physician pulls section 34 in the direction of arrow 42 in FIG. 11. A handle 43 can be provided on section 34 proximally of tube 11 (see FIGS. 10-11). As seen in FIG. 11, the handle position shown in phantom lines shows the handle position when the loop 32 is formed as shown in FIG. 10 and the handle position shown in hard lines in FIG. 11 is the position of the handle when the handle is retracted and the loop 32 is retracted into the bore 12. As seen in FIGS. 4-5, there can be seven (7) strands, cables or wires 22, 23, 24, 25, 26, 27, 28. Each cable, strand, or wire 17, 18, 19 or 22, 23, 24, 25, 26, 27, 28 can form a separate loop distally of tube end 14. Other numbers of cable strands can be used. FIG. 4 compares the smaller profile or outer diameter 31 of the apparatus 10 of the present invention with a prior art snare profile or outer diameter 29 having a tube T and snare cable S. In the larger diameter snare profile or outer diameter 29, the snare S occupies the bore within the tube T. In the prior art, the tube T must be large enough to contain two sections of the snare, side by side, as shown.

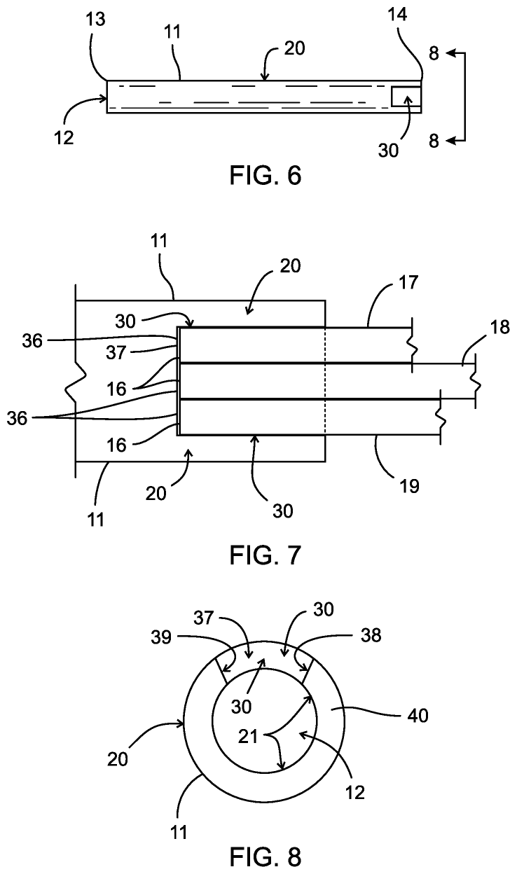

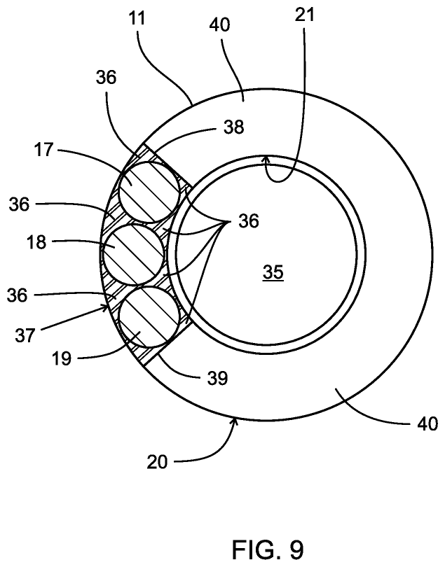

As seen in FIGS. 2, 7 and 9 the distal end 16 of snare member 15 can include three strands, wires or cables 17, 18, 19 that are connected to tube 11 (e.g., welded as shown in FIG. 9) wherein the strands, cables or wires occupy recess or cut-out 30 (see FIGS. 6-8).

As seen in FIGS. 2, 4 and 5 a majority or all of the diameter of each cable or wire 17-19, 22-28 is located in between outer surface 20 and inner surface 21 of outer tube 11. There is thus more room in bore 12 of tube 11 available for that part of snare member 15 that is proximal of snare loop or loops 32. Proximal section 34, that portion of the snare or member that does not form the loop 32, can be a solid wire or hypotube. FIG. 3 illustrates the position of wires or cables 17, 18, 19 that form snare loop or loops 32 at a position spaced distally away from tube 11 distal end 14, that view being a sectional view taken along lines 3-3 of FIG. 1. In FIG. 3, the more proximal section of wires or cables 17, 18, and 19 are not shown in the bore 12 for purposes of clarity. The position of wires or cables 17, 18, and 19 inside the bore can be seen in FIG. 2.

The cut-out or recess 30 can extend between tube outer surface 20 and tube inner surface 21 as seen in FIG. 8. The cut-out or recess 30 is preferably open ended at tube distal end 14 as seen in FIGS. 6-7. A surface 37 is preferably an arcuate surface that defines a stop for strands or cables 17, 18, 19 in FIGS. 2 and 9 (or 22-28 in FIGS. 4-5) (see FIGS. 7-8). Cut-out or recess 30 forms longitudinal surfaces 38, 39 (see FIG. 8).

As seen in FIG. 9, welds 36 connect strands 17 and 19 to strand 18. Welds 36 also may connect strands 17, 18, 19 to arcuate surface 37. Welds 36 join strand 17 to surface 38 and strand 19 to surface 39 (see also FIG. 7 showing distal end of snare member 16 joined to arcuate surface 37 via weld 36). Strands 17, 18, 19 or 22-28 can also be bonded to outer tube 11 at cut-out or recess 30 using soldering or brazing. Optionally small holes can be machined in the tube wall (e.g. using micro-EDM), or small spaces (e.g. like a castle tower top, crenels) and snare strands can be inserted and welded

As seen in FIGS. 2, 4, 5 and 9, a majority or all of the diameter of each strand 17, 18, 19 (or 22-28) is between outer surface 20 and inner surface 21 of outer tube 11. However, each strand 17-19 or 22-28 could have a diameter that is larger than tube wall thickness, but wherein the particular strand 17-19 or 22-28 occupies all or a majority of the thickness of tube 11 wall. Also, small holes can “extend” beyond the tube's wall (and then some section of strands may extend beyond either the tube inner diameter, outer diameter, or both.

Without limitation, the apparatus may be further characterized by one or more of the following statements:

Statement 1: An endovascular snare apparatus, comprising:
a) an outer tube having a proximal end, a distal end, an outer surface, an inner surface, a tube lumen having a central longitudinal axis and an outer tube wall in between the inner surface and the outer surface; and
b) a snare member having a snare proximal section and including one or more snare wires or cables that occupy the tube lumen, said one or more snare wires or cables having a snare proximal end and a snare distal end;
wherein each of said one or more snare wires or cables has an outer diameter;
wherein a majority of the outer diameter of each of said one or more snare wires or cables is positioned in between the inner surface and the outer surface at a connection of the snare distal end to the outer tube;
wherein at said connection, a portion of said snare member is extendable distally of said tube distal end to form a snare loop; and
wherein the snare proximal section extends through said tube lumen to said tube proximal end, wherein said loop is extendable or retractable by manipulating the snare proximal section.

Statement 2: The endovascular snare apparatus of statement 1 further comprising a recess in the outer tube distal end, wherein the snare distal end occupies said recess at said connection.

Statement 3: The endovascular snare apparatus of statement 2 wherein the recess of the outer tube has small holes or spaces and the snare member is bonded to the outer tube at said small holes or spaces.

Statement 4: The endovascular snare apparatus of statement 2 or 3 wherein the snare member is bonded to the outer tube at said recess with a weld, adhesive or brazing or soldering methods.

Statement 5: The endovascular snare apparatus of any one of statements 1-4, wherein the snare member includes multiple strands.

Statement 6: The endovascular snare apparatus of statement 5 wherein said snare member includes between three and nineteen strands, preferably between three and seven strands.

Statement 7: The endovascular snare apparatus of statement 5 or 6 wherein each strand extends to said outer surface at said connection and/or to said inner surface at said connection.

Statement 8: The endovascular snare apparatus of any one of statements 1-7 wherein the snare proximal section is a single strand or a tube.

Statement 9: An endovascular snare apparatus, comprising:
a) an outer tube having a tube proximal end, a tube distal end, an outer surface, an inner surface, an outer tube lumen having a central longitudinal axis, a lumen interval diameter, and a longitudinal axis and an outer tube wall in between the inner surface and the outer surface; and
b) a snare member having a snare proximal section and including one or more snare wires or cables that occupy the outer tube lumen, said one or more snare wires or cables having a proximal end, a distal end, and an outer diameter;
wherein the snare member has a snare member proximal end and a snare member distal end;
wherein a majority of the outer diameter of each of said one or more snare wires or cables is positioned in between the inner surface and the outer surface at a connection of the snare member distal end to the outer tube;
wherein at said connection, a portion of said snare member extends distally of said tube distal end to form a snare loop, said loop comprised of multiple strands that each form a separate single strand loop;
wherein the snare proximal section extends through said outer tube lumen to said tube proximal end, wherein said loop is extendable or retractable by manipulating the snare proximal section; and
wherein said snare proximal section has a diameter larger than half the diameter of said outer tube lumen.

Statement 10: The endovascular snare apparatus of statement 9 wherein said snare member includes between three and nineteen strands.

Statement 11: The endovascular snare apparatus of statement 9 or 10 further comprising a recess in the outer tube distal end, wherein the snare member distal end occupies said recess.

Statement 12: The endovascular snare apparatus of any one of statements 9-11 wherein the snare member is bonded to the outer tube at said recess with a weld, adhesive or brazing or soldering methods.

Statement 13: The endovascular snare apparatus of any one of statements 9-12 wherein each strand extends to said outer surface at said connection and/or to said inner surface at said connection.

Statement 14: The endovascular snare apparatus of any one of statements 9-13 wherein each strand is generally parallel to said longitudinal axis at said connection.

Statement 15: The endovascular snare apparatus of any one of statements 12-14 wherein multiple strands are bonded to each other and to said tube wall at said connection.

Statement 16: An endovascular snare apparatus, comprising:
a) an outer tube having a tube proximal end, a tube distal end, an outer surface, an inner surface, an outer tube lumen having a central longitudinal axis and an outer tube wall in between the inner surface and the outer surface; and
b) a snare member having a snare proximal section and including one or more snare wires or cables that occupy the outer tube lumen, said one or more snare wires or cables having a proximal end, a distal end, and an outer diameter;
wherein the snare member has a snare member proximal end and a snare member distal end;
wherein a majority of the outer diameter of each of said one or more snare wires or cables is positioned in between the inner surface and the outer surface at a connection of the snare member distal end to the outer tube;
wherein at said connection, a portion of said snare member extends distally of said tube distal end to form a snare loop, said snare loop comprised of multiple strands, each strand forming a separate single strand loop;
wherein the snare proximal section extends through said outer tube lumen to said tube proximal end, wherein said loop is extendable or retractable by manipulating the snare proximal section; and
wherein each strand extends a majority of the distance between said inner surface and said outer surface.

Statement 17: The endovascular snare apparatus of statement 16 wherein the snare proximal section has a transverse cross section that occupies between 50-100% percent of the outer tube lumen.

Statement 18: An endovascular snare apparatus, comprising:
a) an outer tube having a proximal end, a distal end, an outer surface, an inner surface, a tube lumen having a central, longitudinal axis and an outer tube wall in between the inner and outer surfaces;
b) a snare member that includes one or more snare wires or cables that occupy the tube lumen, said one or more snare wires or cables having a snare proximal end and a snare distal end;
c) each snare wire or cable having an outer diameter;
d) wherein a majority of the outer diameter of each snare wire or cable is positioned in between the tube inner and outer surfaces at a connection of the snare distal end to the outer tube;
e) wherein at said connection, a portion of said snare member is extendable distally of said tube distal end to form a snare loop; and
f) a proximal portion of said snare member extending through said tube lumen to said tube proximal end so that a user can manipulate the snare member proximal portion to extend or retract said loop.

Statement 19: The endovascular snare apparatus of statement 18 further comprising a recess in the outer tube distal end, wherein the snare distal end occupies said recess at said connection.

Statement 20: The endovascular snare apparatus of statement 18 wherein the snare member includes multiple strands.

Statement 21: The endovascular snare apparatus of statement 19 wherein the snare member includes multiple strands.

Statement 22: The endovascular snare apparatus of any one or more of statements 19-21 wherein the snare member is bonded to the outer tube at said recess with a weld, adhesive or brazing or soldering methods.

Statement 23: The endovascular snare apparatus of any one or more statements 19-22 wherein there are between three and nineteen strands that comprise said snare member.

Statement 24: The endovascular snare apparatus of any one or more statements 19-23 wherein there are between three and seven strands that comprise said snare member.

Statement 25: The endovascular snare apparatus of any one or more of statements 20-21 wherein each strand extends to said tube outer surface at said connection.

Statement 26: The endovascular snare apparatus of any one or more of statements 20-21 wherein each strand extends to said tube inner surface at said connection.

Statement 27: The endovascular snare apparatus of statement 18 wherein the snare member has a proximal section that is a single strand.

Statement 28: The endovascular snare apparatus of statement 18 wherein the snare member has a proximal section that is a tube.

Statement 29: An endovascular snare apparatus, comprising:
a) an outer tube having a proximal end, a distal end, an outer surface, an inner surface, an outer tube lumen having a central, a lumen interval diameter and a longitudinal axis and an outer tube wall in between the inner and outer surfaces;
b) a snare member that includes one or more snare wires or cables that occupy the tube lumen, said one or more snare wires or cables having a proximal end, a distal end, and an outer diameter;
c) the snare member having proximal and distal ends;
d) wherein a majority of the outer diameter of each snare wire or cable is positioned in between the tube inner and outer surfaces at a connection of the snare member distal end to the outer tube;
e) wherein at said connection, a portion of said snare member extends distally of said tube distal end to form a snare loop, said loop comprised of multiple strands that each form a separate single strand loop;
f) a proximal portion of said snare member extending through said tube bore to said tube proximal end so that a user can manipulate the snare member proximal portion to extend or retract said loop; and
g) said proximal portion having a diameter that is more than half the diameter of the said tube lumen.

Statement 30: The endovascular snare apparatus of statement 29 wherein there are between three and nineteen strands that comprise said snare member.

Statement 31: The endovascular snare apparatus of statement 29 further comprising a recess in the outer tube distal end, wherein the snare member distal end occupies said recess.

Statement 32: The endovascular snare apparatus of statement 31 wherein the snare member is bonded to the outer tube at said recess with a weld, adhesive or brazing or soldering methods.

Statement 33: The endovascular snare apparatus of statement 29 wherein each strand extends to said tube outer surface at said connection.

Statement 34: The endovascular snare apparatus of statement 29 wherein each strand extends to said tube inner surface at said connection.

Statement 35: The endovascular snare apparatus of statement 29 wherein each strand is generally parallel to said tube bore longitudinal axis at said connection.

Statement 36: The endovascular snare apparatus of statement 32 wherein multiple strands are bonded to each other and to said tube wall at said connection.

Statement 37: An endovascular snare apparatus, comprising:
a) an outer tube having a proximal end, a distal end, an outer surface, an inner surface, an outer tube lumen having a central, longitudinal axis and an outer tube wall in between the inner and outer surfaces;
b) a snare member that includes one or more snare wires or cables that occupy the tube lumen, said one or more snare wires or cables having a proximal end, a distal end, and an outer diameter;
c) the snare member having proximal and distal ends;
d) wherein a majority of the outer diameter of each snare wire or cable is positioned in between the tube inner and outer surfaces at a connection of the snare member distal end to the outer tube;
e) wherein at said connection, a portion of said snare member extends distally of said tube distal end to form a snare loop, said loop comprised of multiple strands, each strand forming a separate single strand loop;
f) a proximal portion of said snare member extending through said tube bore to said tube proximal end so that a user can manipulate the snare member proximal portion to extend or retract said loop; and
g) wherein each strand extends a majority of the distance between said tube inner and outer surfaces.

Statement 38: The endovascular snare apparatus of statement 37 wherein the snare member proximal portion has a transverse cross section that occupies between 50-100% percent of the tube lumen.

Statement 39: The endovascular snare apparatus of statement 19 wherein the recess of the outer tube has small holes or spaces and the snare member is bonded to the outer tube at said small holes or spaces.

The following is a list of parts and materials suitable for use in the present invention:

PARTS LIST

| PART NUMBER | DESCRIPTION |
|---|---|
| 10 | endovascular snare apparatus |
| 11 | outer tube / tube / hypotube |
| 12 | bore/lumen |
| 13 | tube proximal end |
| 14 | tube distal end |
| 15 | snare member |
| 16 | snare member distal end/snare member fixed |
| | end/snare member free end |
| 17 | wire/cable/strand |
| 18 | wire/cable/strand |
| 19 | wire/cable/strand |
| 20 | tube outer surface |
| 21 | tube inner surface |
| 22 | wire/cable/strand |
| 23 | wire/cable/strand |
| 24 | wire/cable/strand |
| 25 | wire/cable/strand |
| 26 | wire/cable/strand |
| 27 | wire/cable/strand |
| 28 | wire/cable/strand |
| 29 | snare profile |
| 30 | recess/cutout/void space |
| 31 | outer diameter |
| 32 | snare loop |
| 33 | length of snare section that is proximal of loop |
| 34 | snare proximal section |
| 35 | mandrel |
| 36 | weld |
| 37 | arcuate surface |
| 38 | longitudinal surface |
| 39 | longitudinal surface |
| 40 | tube wall |
| 41 | arrow |
| 42 | arrow |
| 43 | handle |
| T | tube |
| S | snare cable |

The foregoing embodiments are presented by way of example only; the scope of the present invention is to be limited only by the following claims.

1-39. (canceled)

40. An endovascular snare apparatus, comprising:
an outer tube having a proximal end and a distal end, the outer tube defining a tube lumen extending from the distal end towards to the proximal end, the outer tube defining at least one recess at the distal end;
a snare member having a snare proximal section, the snare member including one or more snare wires or cables, the one or more snare wires or cables having a snare proximal end and a snare distal end;
wherein the snare distal end of each of the one or more snare wires or cables is disposed in and attached to the outer tube at the at least one recesses;
wherein a portion of the snare member is extendable distally of the distal end to form at least one snare loop; and
wherein the snare proximal section extends through the tube lumen to the proximal end, wherein the at least one snare loop is extendable or retractable by manipulating the snare proximal section.

41. The endovascular snare apparatus of claim 40, wherein each of the one or more snare wires or cables exhibit a diameter that is less than a thickness of the outer tube measured between an outer surface and an inner surface of the outer tube.

42. The endovascular snare apparatus of claim 40, wherein the one or more snare wires or cables extends at least one of inwardly past an inner surface of the outer tube or outwardly past an outer surface of the outer tube.

43. The endovascular snare apparatus of claim 40, wherein the one or more snare wires or cables includes a plurality of snare wires or cables.

44. The endovascular snare apparatus of claim 43, wherein the plurality of snare wires or cables includes 3 to 7 snare wires or cables.

45. The endovascular snare apparatus of claim 43, wherein the snare member includes a single wire or tube attached to the snare proximal end of each of the plurality of snare wires or cables.

46. The endovascular snare apparatus of claim 43, wherein the at least one snare loop forms a plurality of snare loops, each of the plurality of snare loops formed by one of the plurality of snare wires or cables.

47. The endovascular snare apparatus of claim 43, wherein the at least one recess includes a plurality of recesses and the snare distal end of each of the plurality of snare wires or cables are disposed in a corresponding one of the plurality of recesses.

48. The endovascular snare apparatus of claim 43, wherein a portion of each of the plurality of snare wires or cables extending from the at least one recess are generally parallel to a longitudinal axis of the tube lumen.

49. The endovascular snare apparatus of claim 43, wherein the distal end of each of the plurality of snare wires or cables are attached together.

50. The endovascular snare apparatus of claim 40, wherein the at least one recess includes a single recess extending completely through a thickness of the outer tube.

51. The endovascular snare apparatus of claim 40, wherein the at least one recess extending from at least one of an outer surface or an inner surface of the outer tube.

52. The endovascular snare apparatus of claim 40, wherein the snare distal end of the one or more snare wires or cables are attached to portions of the outer tube defining the at least one recess with a weld.

53. The endovascular snare apparatus of claim 40, wherein the snare distal end of the one or more snare wires or cables are attached to portions of the outer tube defining the at least one recess with at least one of a braze or solder.

54. A method of using an endovascular snare apparatus, the method comprising:
providing the endovascular snare apparatus, the endovascular snare apparatus including:
an outer tube having a proximal end and a distal end, the outer tube defining a tube lumen extending from the distal end towards to the proximal end, the outer tube defining at least one recess at the distal end;
a snare member having a snare proximal section, the snare member including one or more snare wires or cables, the one or more snare wires or cables having a snare proximal end and a snare distal end;
wherein the snare distal end of each of the one or more snare wires or cables is disposed in and attached to the outer tube at the at least one recesses;
wherein a portion of the snare member is extendable distally of the distal end to form at least one snare loop; and
manipulating the snare proximal section of a snare member to extend or retract the at least one snare loop.

55. The method of claim 54, wherein manipulating the snare proximal section includes moving the snare proximal section of the snare member in a distal direction relative to the outer tube to extend the at least one snare loop.

56. The method of claim 54, wherein manipulating the snare proximal section includes moving the snare proximal section of the snare member in a proximal direction relative to the outer tube to retract the at least one snare loop.

57. The method of claim 54, wherein manipulating the snare proximal section includes manipulating a handle coupled to the snare proximal section.

58. A method of manufacturing an endovascular snare apparatus, the method comprising:
disposing a snare distal end of one or more snare wires or cables in at least one recess formed in a distal end of an outer tube; and
attaching the snare distal end of the one or more snare wires or cables to portions of the outer tube defining the at least one recess;
wherein the outer tube has a proximal end and the distal end, the outer tube defining a tube lumen extending from the distal end towards to the proximal end;
wherein the one or more snare wires or cables form at least a portion of a snare member, the snare member having a snare proximal section, the one or more snare wires or cables having a snare proximal end and the snare distal end;
wherein a portion of the snare member is extendable distally of the distal end to form at least one snare loop; and
wherein the snare proximal section extends through the tube lumen to the proximal end, wherein the at least one snare loop is extendable or retractable by manipulating the snare proximal section.

59. The method of claim 58, wherein:
the one or more snare wires or cables includes a plurality of snare wires or cables
disposing a snare distal end of one or more snare wires or cables in at least one recess formed includes disposing the snare distal end of each of the plurality of snare wires or cables in the at least one recess; and
attaching the snare distal end of the one or more snare wires or cables to portions of the outer tube defining the at least one recess includes attaching the snare distal end of each of the plurality of snare wires or cables to the portions of the outer tube defining the at least one recess and to each other.
